(19) United States
(12) Patent Application Publication
(10) Pub. No.: US 2023/0166077 A1
(43) Pub. Date: Jun. 1, 2023

(54) INJECTION SYSTEMS

(71) Applicant: Mayo Foundation for Medical Education and Research, Rochester, MN (US)

(72) Inventors: Emanuel C. Trabuco, Rochester, MN (US); Jordan D. Miller, Rochester, MN (US); Erika K. Ross, San Mateo, CA (US)

(21) Appl. No.: 17/913,291

(22) PCT Filed: Apr. 13, 2021
(86) PCT No.: PCT/US2021/027059

§ 371 Date: Sep. 21, 2022

Related U.S. Application Data
(60) Provisional application No. 63/011,965, filed on Apr. 17, 2020.

(51) Int. Cl. A61M 25/00

(52) U.S. Cl. CPC A61M 25/0084; A61M 2025/105

(57) ABSTRACT

Devices and methods are described for injecting therapeutics into tissue. For example, some embodiments disclosed in this document describe devices and methods for injecting muscle precursor cells into the sphincter urethrae to treat stress urinary incontinence.

### CROSS-REFERENCE TO RELATED APPLICATIONS

This application claims the benefit of U.S. Provisional Application Serial No. 63/011,965, filed Apr. 17, 2020. The disclosure of the prior application is considered part of (and is incorporated by reference in) the disclosure of this application.

### BACKGROUND

### 1. Technical Field

This document relates to devices and methods for injecting therapeutics into tissue. For example, some embodiments disclosed in this document relate to devices and methods for injecting bulking agents, agents that promote muscle regeneration and improved muscle function, and muscle precursor cells into the sphincter urethrae to treat stress urinary incontinence. Broader embodiments disclosed in this document relate to devices and methods for injecting bulking agents, agents that promote muscle regeneration and improved muscle function, and muscle precursor cells into other structures in which compromised sphincter function results in impaired physiological function (including but not limited to treatment of the gastro-esophageal sphincter for treatment of reflux).

### 2. Background Information

Urinary incontinence afflicts between 10 to 40% of adult women in the United States. Stress urinary incontinence (SUI), the involuntary loss of urine associated with physical activity due to impaired function of the urinary sphincter, is the most common form representing approximately one-third of cases. Although not life threatening, urinary incontinence greatly impacts a woman’s quality of life; with similar Health Utility Index scores being reported among women seeking treatment for stress urinary incontinence (0.67-0.73) and community dwelling women with other chronic, debilitating illnesses such as stroke (0.67), cancer (0.80), diabetes mellitus (0.74) and back pain (0.80).

Sphincter dysfunction is not an uncommon root cause of a number of other medical conditions and surgical complications, and includes: gastro-esophageal reflux, fecal incontinence, urinary incontinence in men after radical prostatectomy, and a host of other conditions.

About 3.4 million men in the United States have urinary incontinence. In men, urinary incontinence can be brought on by various medical conditions such as enlarged prostate, diabetes, and Parkinson’s disease. It can also be common after some types of prostate surgery. Urinary incontinence is a treatable condition.

### SUMMARY

This document describes devices and methods for injecting therapeutics into tissue. For example, some embodiments disclosed in this document describe devices and methods for injecting bulking agents, agents that promote muscle regeneration and improved muscle function, and muscle precursor cells into the sphincter urethrae to treat stress urinary incontinence. The devices and methods described herein can be used to treat both men and women.

In one aspect, an injection device is described herein. In one embodiment, the injection device includes a handle, a shaft extending distally from the handle, and two or more hypodermic needles. Each of the hypodermic needles can be reconfigured between: (i) a first position that is fully within the shaft and (ii) a second position in which a distal tip portion of the hypodermic needle extends radially from the shaft.

Such an injection device may optionally include one or more of the following features. The handle may have an actuator to control the radial movement of the needles between the first and second positions. The injection device may also include a ruled indicator showing radial extension positions of the hypodermic needles. The injection device may include four hypodermic needles. In some embodiments, the four hypodermic needles are all radially extendable from the shaft to be within an envelope of less than 180°. The injection device may also include an expandable balloon attached to the shaft. In some embodiments, the balloon is distal of the hypodermic needles. The hypodermic needles may be manually translatable along a longitudinal axis of the shaft. In some embodiments, the handle has an actuator to control the translation of the hypodermic needles along the longitudinal axis of the shaft. The hypodermic needles may also be actuated to emerge radially from the shaft at multiple positions along the longitudinal axis of the shaft. The injection device may also include an indicator that shows a position of the hypodermic needles along the longitudinal axis of the shaft. In some embodiments, the hypodermic needles may be extended radially within a 0 mm to 5 mm range from an outer surface of the shaft. The shaft may have a measuring scale proximal of the hypodermic needles.

In another aspect, this disclosure is directed to a method of treating urinary incontinence. In some embodiments, the method includes: (i) inserting a shaft of an injection device into a urethra so that a distal tip portion of the shaft resides within a bladder; (ii) expanding an expandable member that is attached to the distal tip portion of the shaft; (iii) applying proximal traction of the shaft to cause the expandable member to abut an inner wall of the bladder around an opening to the urethra; (iv) while the proximal traction is being applied, measuring a length of the urethra using a scale on the shaft; (v) extending two or more needles laterally from the shaft so that distal tip portions of the two or more needles puncture and extend through an inner wall of the urethra; and (vi) while the two or more needles are extending through the inner wall of the urethra, injecting a therapeutic via the two or more needles.

Such a method may optionally include one or more of the following features. A mid-point of the urethra may be determined based on the measured length of the urethra. The two or more needles may be extended to puncture the inner wall starting at the mid-point. The two or more needles may be moved longitudinally along the shaft and multiple injections may be delivered at multiple locations longitudinally along the urethra. In some embodiments, the therapeutic is a purified exosome product (PEP).

Particular embodiments of the subject matter described in this document can be implemented to realize one or more of the following advantages. In some embodiments, the devices and methods can be used to treat stress urinary incontinence. The devices include features to help a clinician verify target locations for a series of injections of therapeutics so that the injections reach the sphincter urethrae as desired. In some embodiments, stress urinary incontinence can be treated in a minimally invasive fashion using the devices and methods provided herein. Broader embodiments disclosed in this document relate to devices and methods for injecting bulking agents, agents that promote muscle regeneration and improved muscle function, and muscle precursor cells into other structures in which compromised sphincter function results in impaired physiological function in a minimally invasive fashion using devices and methods described herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of a female urethra and an example mid-urethral drug delivery device.

FIG. 2 is a schematic diagram of the mid-urethral drug delivery device inserted into the female urethra.

FIG. 3 is a schematic diagram of the mid-urethral drug delivery device inserted into the female urethra and a balloon member of the device in an inflated state.

FIG. 4 is a schematic diagram of mid-urethral drug delivery device with drug delivery needles of the device in a laterally extended configuration.

FIG. 5 is an enlarged view of the working end of the device.

FIG. 6 is a transverse view of a shaft of the mid-urethral drug delivery device showing a first example orientation of the drug delivery needle’s orientation radially extending from the shaft.

FIG. 7 is another transverse view of the shaft of the mid-urethral drug delivery device showing a second example orientation of the drug delivery needle’s orientation radially extending from the shaft.

FIG. 8 is a schematic diagram of the mid-urethral drug delivery device as it is injecting a therapeutic (e.g., PEP matrix, bulking agent, etc.) into the urethral sphincter muscles.

FIG. 9 is a schematic diagram of the mid-urethral drug delivery device after actuation of a valve used to expand and contract the balloon member.

FIG. 10 is a schematic diagram depicting the removal of the mid-urethral drug delivery device from the female urethra.

FIG. 11 is a flowchart depicting a method of treating urinary incontinence using a mid-urethral drug delivery device as described herein.

Like reference numbers represent corresponding parts throughout.

### DETAILED DESCRIPTION

This document describes devices and methods for injecting therapeutics into tissue. For example, some embodiments disclosed in this document describe devices and methods for injecting muscle precursor cells into the sphincter urethrae to treat stress urinary incontinence.

While the devices and methods disclosed herein are described in the context of trans-urethral injections to treat female urinary incontinence, it should be understood that the devices and methods (or minor modifications thereof) can be implemented in many other contexts with beneficial efficaciousness. For example, in some embodiments the devices and methods described herein can be implemented for treating peri-intestinal structures, perivascular structures, male incontinence, and in other contexts in which injections of therapeutic substrates are beneficial.

By the new devices and methods disclosed herein, the inventors are implementing innovative therapies utilizing, for example, a purified exosome product (PEP) to recruit local stem cell migration and differentiation (which is distinguished from traditional cell-based regenerative technology). The inventors have discovered that PEP has the capability to induce local MPC cell migration and differentiation to restore external urethral sphincter function. Accordingly, this treatment offers a novel and less costly treatment option for SUI.

This disclosure describes a delivery system for any therapeutic substances including bulking agents, cell based therapies, or other compounds, substrates, or therapeutic substances, without limitation. In some embodiments, the delivery system can be used to measure the length of the urethra, and to deliver between 25 and 1000 micro liters of PEP matrix at positions along the middle of the urethra. The delivery system is designed to cause minimal discomfort and to be used in the outpatient setting obviating the need to go to the operating room. Measuring the length of the urethra is beneficial as the external urethral sphincter is not readily visualized, and the known location is at the mid-point of the urethra. The devices described herein have retractable hypodermic needles that are deployed at the set distance (after the measurement of length is obtained) to deliver bilateral therapeutic substance to the external urethral sphincter.

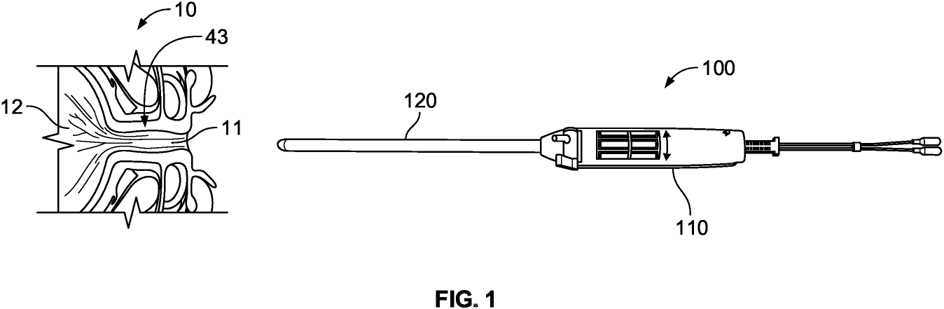

FIG. 1 shows a cross-section of a female urethral area 10 and an example mid-urethral drug delivery device 100. The mid-urethral drug delivery device 100 can be used by a clinician to treat urinary incontinence as described further herein. For the purposes of the descriptions herein, the components of the mid-urethral drug delivery device 100 to the right in FIG. 1 are deemed as oriented proximal to the user (e.g., the clinician), and components to the left are deemed as oriented distally to the user.

The mid-urethral drug delivery device 100 includes a handle 110 and a shaft 120 that distally extends from the handle 110. To perform the urinary incontinence treatment, the shaft 120 of the mid-urethral drug delivery device 100 is designed to be inserted into the urethra 11 and partially advanced into the bladder 12, as described further below.

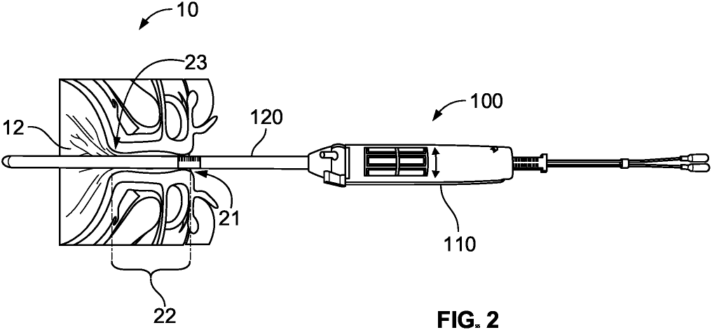

Referring also to FIG. 2, the shaft 120 of the mid-urethral drug delivery device 100 can be inserted into the urethra 11. The distal blunt tip portion of shaft 120 is inserted through a urethral opening 21, along a length 22 of the urethra 11, through a neck 23 of the bladder 12, and into the bladder 12.

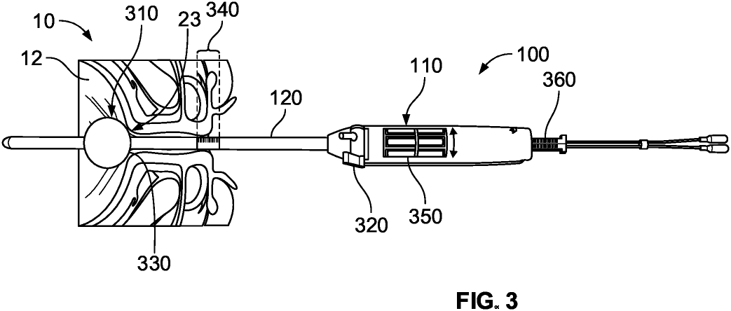

Referring also to FIG. 3, an expandable balloon 310 may be inflated within the bladder 12. The balloon 310 is attached to the distal tip portion of the shaft 120. The inflation of the expandable balloon 310 is controlled by the clinician using a flow valve 320 that can be coupled to the handle 110. The flow valve 320 can be actuated to direct fluid through the luminal portion of the shaft 120 and cause the expandable balloon 310 to inflate. In some embodiments, a mechanical expendable element is used instead of the balloon 310.

With the balloon 310 in its expanded state, the mid-urethral drug delivery device 100 may then be held in place by the clinician with light tension applied via the handle 110 in a proximal direction such that the proximal base 330 of the expandable balloon 310 is abutting the neck 23 of the bladder 12. Accordingly, the balloon 310 can be used in this manner to positively register the position of the mid-urethral drug delivery device 100 in relation to the female urethral area 10.

While the balloon 310 is held against the neck 23 of the bladder 12, the length 22 of the urethra 11 is then visually measured by the clinician using a section of ruled markings 340 on the shaft 120. The average length of the female urethra is approximately 4 cm ± 1 cm and the ruled markings 340 may be positioned proximally along the shaft 120 from the base 330 of the expandable balloon 310 such that this range (e.g., ≥ 3 cm, ≤ 5 cm) can be visualized in millimeter increments.

Measuring the length 22 of the urethra 11 is performed by the clinician to determine, for example, a mid-point of the urethra 11. The use of the measurement data is described further below.

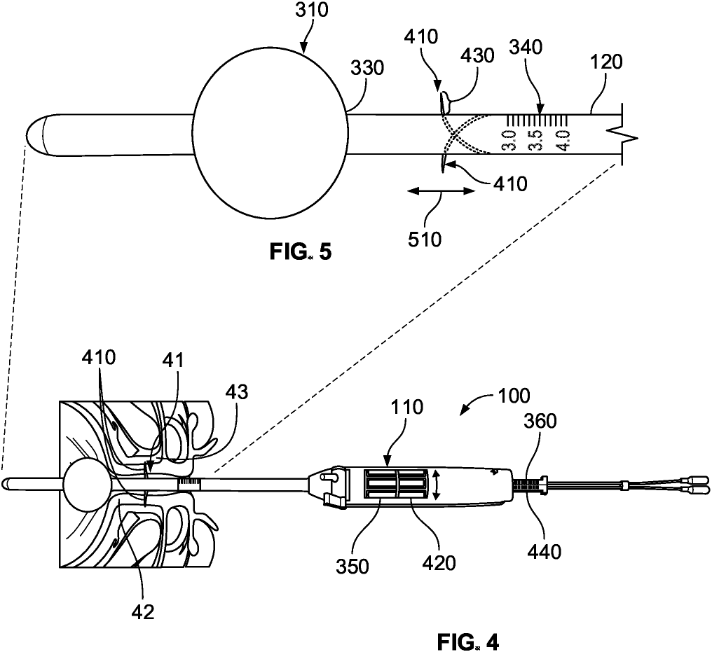

Referring also to FIGS. 4 and 5, the mid-urethral drug delivery device 100 includes two or more injection needles 410 that can be used to deliver a therapeutic substance. The clinician can control the needles 410 to make them radially extend from the shaft 120 or be contained within the shaft 120. As the shaft 120 is advanced into the urethra 11, the needles 410 are kept contained within the shaft 120. Thereafter, the needles 410 can be actuated by the clinician to radially extend (so as to puncture the surface of the tissue of the urethra 11, and to extend into a urethral sphincter 43 around the urethra 11).

The needles 410 can also be selectively positioned at various locations along the longitudinal axis of the shaft 120. The clinician can control the positioning of the needles 410 along the longitudinal axis of the shaft 120.

Once a measurement of the length 22 of the urethra 11 has been obtained using the ruled markings 340, the position of the needles 410 along the longitudinal axis of the shaft 120 may be adjusted by the clinician until desired placement is achieved. In some embodiments, the position of the needles 410 along the longitudinal axis of the shaft 120 may be adjusted by the clinician by rotating an axial position dial 350 until desired placement is achieved. In some embodiments, other types of adjustment mechanisms are used. In the depicted embodiment, the axial position dial 350 used to control the axial location of the needles 410 may be mechanically connected to an axial location indicator 360. The axial location indicator 360 may be located on the proximal end of the handle 110 and may be used to indicate the axial location of the needles along the longitudinal axis of the shaft 120. The axial location indicator 360 may be ruled, for example, in millimeter increments. The axial location indicator 360 may also have a first marking that denotes the maximal distal position of the needles 410, and a second marking proximal of the first marking denoting the maximal proximal position of the needles 410. The axial location indicator 360 may further have a third marking, between the first and second markings, to denote the average center position of the needles 410 in the urethra 11. In some embodiments, the marks may be read relative to the proximal end of the handle 110.

As shown in FIG. 4, the clinician may then cause the needles 410 to be radially extended from the walls of the shaft 120 by rotating a needle extension dial 420 that is rotatably coupled to the handle 110. This bilaterally advances the needles 410 through the muscosa 41 and smooth muscle 42 of the urethra 11 and into the urethral sphincter 43.

The radial extension dial 420 may be used by the clinician to control the radial extension 430 of the needles 410 through a range 0 cm to 5 cm, or ≥ 5 cm (e.g., 5 cm, 5.1 cm, 5.2 cm, etc.). In some embodiments, the maximal radial extension 430 of the needles may be approximately 5 cm.

The needle extension dial 420 used to control the radial extension 430 of the needles 410 may also be mechanically connected to a radial extension indicator 440. The needle extension dial 420 may be located on the proximal end of the handle 110 and may be used to indicate the radial extension 430 of the needles 410 from the shaft 120. The needle extension indicator 360 may be ruled in millimeter increments. The needle extension dial 420 may also have a first marking that denotes the maximal radial extension 430 of the needles, and a second marking proximal of the first marking denoting the maximal radial retraction of the needles. The needle extension dial 420 may further have a third marking, between the first and second markings, to note the center radial extension 430 of the needles 410 in the shaft 120 that corresponds with the tip of the needles 410 being in plane with the walls of the shaft 120. In some embodiments, the marks may be read relative to the proximal end of the handle 110.

FIG. 5 is an enlarged view of the distal end of the shaft 120. The expandable balloon 310 is depicted in its expanded configuration such that its proximal base 330 is defined. The at least two needles 410 (e.g., 2, 4, etc.) are depicted in their radially-extended configuration (i.e., being extended from the shaft by a distance of the radial extension 430). Also, and the ruled markings 340 for measuring the length 22 of the urethra 11 are depicted.

In some embodiments, the axial position of the needles 410 along the longitudinal axis of the shaft 120 may be adjusted by the clinician across an axial distance range 510 of 1.4 cm to 2.4 cm (e.g. ≥1.4 cm, ≤ 2.4 cm) as measured from the from the proximal base 330 of the expandable balloon 310 (to provide a range of needle advancement locations into the urethral sphincter 43). The axial location indicator 440 may be ruled to cover the complete axial range 510. In some embodiments, the ruled markings 340 are located approximately 3.5 cm proximal from the base 330 of the expandable balloon 310.

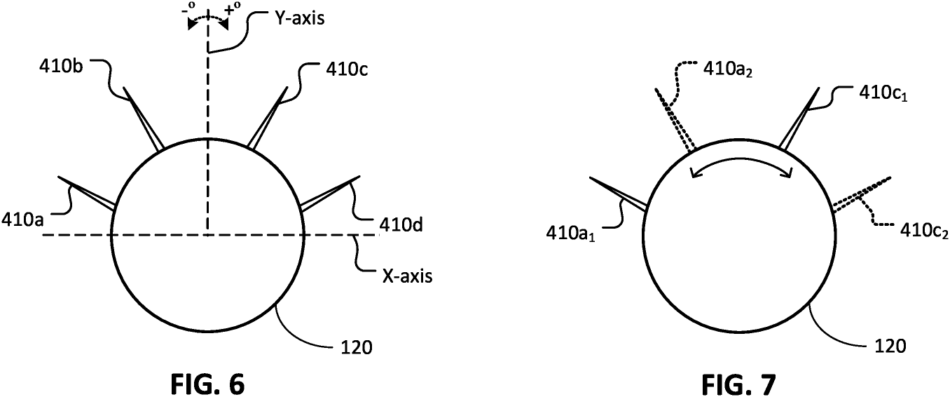

FIG. 6 shows a transverse cross-sectional view of an example embodiment of the shaft 120 and the needles 410. The depicted embodiment includes four needles 410 (indicated individually here as 410a, 410b, 410c, and 410d). While the needles 410 can be located so as to radially extend from the shaft 120 at any locations around the transverse circumference of the shaft 120, in the depicted embodiment all four needles 410 are within a 180° envelop. More particularly, in the depicted embodiment a first needle 410a is located at about -60° relative to the Y-axis. A second needle 410b is located at about -30° relative to the Y-axis. A third needle 410c is located at about 30° relative to the Y-axis. A fourth needle 410d is located at about 60° relative to the Y-axis. Hence, the total arc between the first needle 410a and the fourth needle 410d is about 120°.

FIG. 7 shows a transverse cross-sectional view of another example embodiment of the shaft 120 and the needles 410. The depicted embodiment includes two needles 410, that is: (i) a first needle 410a that can be positioned in a first location 410a₁ and a second location 410a₂, and (ii) a second needle 410c that can be positioned in a first location 410c₁ and a second location 410c₂. In the depicted embodiment, the first needle 410a and the second needle 410c are separated by an arc of about 90°.

In one example usage technique, the two needles 410a and 410c are actuated to radially extend into their first respective locations 410ai and 410ci. Injections of a therapeutic substance via the needles 410a and 410c can be then delivered. Thereafter, the needles 410a and 410c can be radially withdrawn back into the confines of the shaft 120. Next, the clinician can rotate the shaft 120 by about 30 °. In that position, the clinician can cause the needles 410a and 410c to radially extend into their second respective locations 410a₂ and 410c₂. Then, while the needles 410a and 410c are in their radially extended configurations, second injections of the therapeutic substance can be delivered. Accordingly, it can be envisioned that the embodiment of FIG. 7 (having just the two needles 410a and 410c) can be used in a two-step injection process to deliver the same four injections that the embodiment of FIG. 6 can deliver in a single injection step (and without having to rotate the shaft 120).

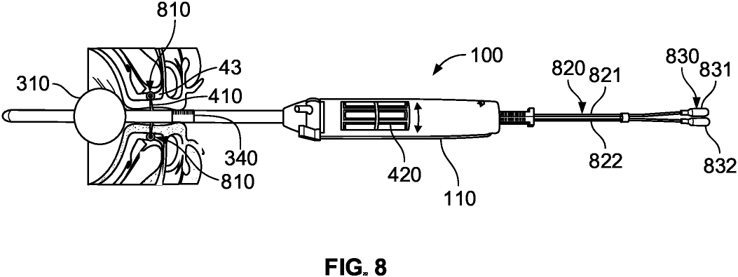

FIG. 8 shows an example injection 810 of a therapeutic substance (e.g., PEP matrix) into the urethral sphincter 43. After the expandable balloon 310 has been inflated and a urethral length 22 measurement taken using the ruled markers 340, the needles 410 may be actuated to extend radially from the shaft 120 (using the needle extension dial 420) to enter into the urethral sphincter 43. Upon advancing to a desired position within the radial extension 430 range (e.g., 1 cm, 2 cm, 3 cm, 4 cm, 5 cm, etc.), an injection 810 of the therapeutic substance (e.g., PEP matrix) may then be administered.

Extending from the proximal end of the handle 110 are a pair of delivery tubes 820, including a right delivery tube 821 and a left delivery tube 822. These delivery tubes 820 continue longitudinally through the body of the handle 110, and terminate at fluid connections to the respective needles 410. Connecting to the proximal end of the delivery tubes 820 are a pair of injection ports 830, including a right injection port 831 and a left injection port 832. In some embodiments, a preferred total volume (V_{T}) of PEP matrix 810 administered may be between 200 mL and 500 mL (e.g. 500 mL ≥ V_{T} ≥ 200 mL), but any other desired volumes, such as between 100 mL and 1000 mL (e.g., 1000 mL ≥ V_{T} ≥ 100 mL), may also be administered.

After advancing the needles 410 and administering a first injection 810 at a desired depth within the urethral sphincter 43, the needles 410 may then be retracted into the shaft 120 by actuating the extension dial 420. If a second injection is desired, the needles 410 may be moved longitudinally along the shaft 120 to a new position along the urethral length 22 by actuating the axial position dial 350. Then, the needles 410 can be re-extended into the urethral sphincter 43 and a second injection 810 administered. Thereafter, the needles 410 can again retracted into the shaft 120. This process may be repeated up to four or more times (e.g., 1, 2, 3, 4, 5, 6, etc.) at unique sites located axially along the urethral length 22. In some embodiments, the injections may be separated by spacings of between 5 mm and 10 mm apart, or 2 mm and 6 mm apart, without limitation.

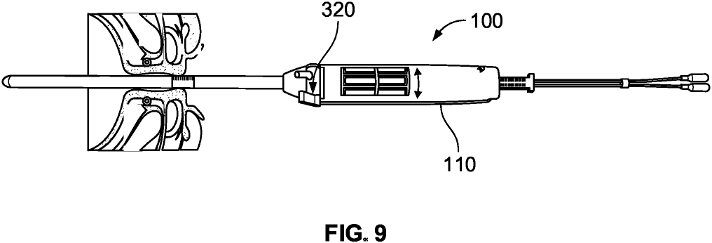

Referring also to FIG. 9, once the injection 810, or series of injections, is complete, the expandable balloon 310 may be deflated by removing the inflating liquid from the expandable balloon 310 through the operation of the flow valve 320.

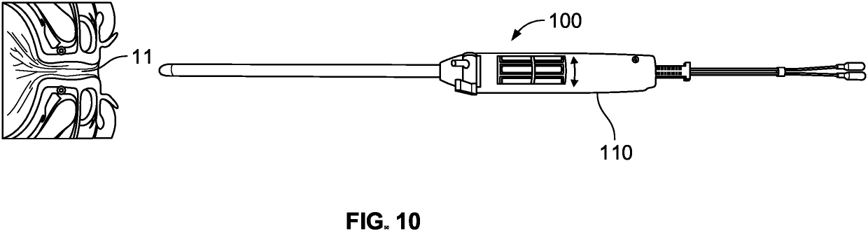

FIG. 10 shows the mid-urethral drug delivery device 100 after removal from from the urethra 11 by pulling the handle 110 proximally until the mid-urethral drug delivery device 100 is fully clear of the urethra 11.

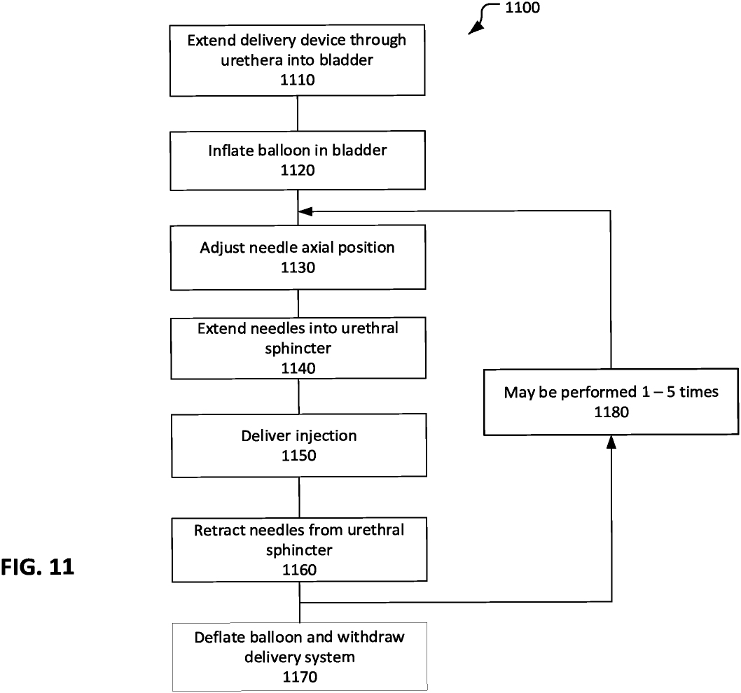

FIG. 11 is a flow diagram of a method 1100 detailing the steps 1110 through 1180 a clinician user may go through to administer one or more injections of a therapeutic substance using the mid-urethral drug delivery device 100. The steps 1110 through 1180 of the method 1100 are described in detail in reference to FIGS. 1-10.

### Additional Features, Embodiments, and Implementations

The general concepts described herein in reference to FIGS. 1 through 11 can be used to describe broader applications of the inventive aspects to other uses/tissues. For example:

FIG. 1 could be a schematic of a tubular structure with an externally accessible orifice and specified region of desired substrate delivery.

FIG. 2 could be a schematic of the device inserted into the tubular structure.

FIG. 3 could show the inflation of the balloon at a distal point where there is a structure that enables appropriate stabilization of the balloon and confirmation of positioning.

FIG. 4 would depict the placement of the delivery of the device with the needle orientation extending radially into the approximate region of the sphincter.

FIG. 5 could be an enlarged view of the working portion of the device at the expected distance of the sphincter.

FIG. 6 could be a transverse view of a shaft of the drug delivery device showing a first example orientation of the drug delivery needle’s orientation radially extending from the shaft.

FIG. 8 could be a schematic diagram of the drug delivery device as it is injecting a therapeutic (e.g., PEP matrix, bulking agent, etc...) into the site of the dysfunctional sphincter muscles.

FIG. 9 could be a schematic diagram of the drug delivery device after actuation of a valve used to expand and contract the balloon member.

FIG. 10 could be a schematic diagram depicting the removal of the drug delivery device by retraction out of the orifice into which entry/access was enabled.

FIG. 11 could be a flowchart depicting a method of treating sphincter dysfunction using a device as described herein.

The devices, systems, and methods described herein can be used to treat conditions such as, but not limited to, gastro-esophageal reflux, fecal incontinence, peri-vascular diseases (including malformations and other conditions), spontaneous male urinary incontinence, post-operative male urinary incontinence (e.g., following radical prostatectomy), and esophageal cancer by delivering targeted anti-cancer treatments to specified regions of the esophagus.

Certain features may be increased or decreased in size or characteristics (in proportion to one another or out of proportion to one another), including but not limited to the shaft diameter, balloon size, shaft length, needle size (including but not limited to length, number of needles, outer diameter, curvature, and lumen diameter) to obtain optimal therapeutic outcomes with any variety of therapeutic substrates applied or tissues/structures targeted.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. An injection device comprising:
a handle;
a shaft extending distally from the handle; and
two or more hypodermic needles that can each be reconfigured between: (i) a first position that is fully within the shaft and (ii) a second position in which a distal tip portion of the hypodermic needles extends radially from the shaft.

2. The device of claim 1, wherein the handle has an actuator to control the radial movement of the needles between the first and second positions.

3. The device of claim 1, further comprising a ruled indicator showing radial extension positions of the hypodermic needles.

4. The device of claim 1, wherein the device comprises four hypodermic needles.

5. The device of claim 4, wherein the four hypodermic needles are all radially extendable from the shaft to be within an envelope of less than 180°.

6. The device of claim 1, further comprising an expandable balloon attached to the shaft.

7. The device of claim 6, wherein the balloon is distal of the hypodermic needles.

8. The device of claim 1, wherein the hypodermic needles are manually translatable along a longitudinal axis of the shaft.

9. The device of claim 8, wherein the handle has an actuator to control the translation of the hypodermic needles along the longitudinal axis of the shaft.

10. The device of claim 8, wherein the hypodermic needles can be actuated to emerge radially from the shaft at multiple positions along the longitudinal axis of the shaft.

11. The device of claim 8, wherein the device further comprises an indicator that shows a position of the hypodermic needles along the longitudinal axis of the shaft.

12. The device of claim 1, wherein the hypodermic needles may be extended radially within a 0 mm to 5 mm range from an outer surface of the shaft.

13. The device of claim 1, wherein the shaft has a measuring scale proximal of the hypodermic needles.

14. A method of treating urinary incontinence, the method comprising:
inserting a shaft of an injection device into a urethra so that a distal tip portion of the shaft resides within a bladder;
expanding an expandable member that is attached to the distal tip portion of the shaft;
applying proximal traction of the shaft to cause the expandable member to abut an inner wall of the bladder around an opening to the urethra;
while the proximal traction is being applied, measuring a length of the urethra using a scale on the shaft;
extending two or more needles laterally from the shaft so that distal tip portions of the two or more needles puncture and extend through an inner wall of the urethra; and
while the two or more needles are extending through the inner wall of the urethra, injecting a therapeutic via the two or more needles.

15. The method of claim 14, wherein a mid-point of the urethra is determined based on the measured length of the urethra.

16. The method of claim 15, wherein the two or more needles are extended to puncture the inner wall starting at the mid-point.

17. The method of claim 14, wherein the two or more needles are moved longitudinally along the shaft and multiple injections are delivered at multiple locations longitudinally along the urethra.

18. The method of claim 14, wherein the therapeutic is a purified exosome product (PEP).
